(19) United States
(12) Patent Application Publication
(10) Pub. No.: US 2018/0075207 A1
(43) Pub. Date: Mar. 15, 2018

(54) Systems and Methods For Performing Health Risk Assessments and Risk Stratification On A Patient Population

(71) Applicant: Solera Health, Inc., Phoenix, AZ (US)

(72) Inventor: Brenda Schmidt, Phoenix, AZ (US)

(21) Appl. No.: 15/783,896

(22) Filed: Oct. 13, 2017

Related U.S. Application Data
(63) Continuation-in-part of application No. 14/808,956, filed on Jul. 24, 2015.
Application No. 14/808,956 is a continuation-in-part of application No. 15/049,723, filed on Feb. 22, 2016.

(51) Int. Cl. G06F 19/00; A61B 5/00

(52) U.S. Cl. CPC G06F 19/3431; G06F 19/3443; A61B 5/7275; G06Q 50/22; G06F 19/3487; G06F 19/363; G06F 19/3437

(57) ABSTRACT

Methods performed by a computer system include the automated processing of patient risk stratification for a patient population, calculating wellness scores for individual patents, assigning a risk level to each patient, and enrolling high risk patients in appropriate disease prevention programs.

### CROSS-REFERENCES TO RELATED APPLICATIONS

This patent application is a continuation-in-part of U.S. patent application Ser. No. 14/808,956 filed Jul. 24, 2015, and published on Jan. 26, 2017, as U.S. Patent Publication No. 2017/0024544, which is incorporated by reference herein.

This patent application is a continuation-in-part of U.S. patent application Ser. No. 15/049,723 filed Feb. 22, 2016, and published on Jan. 26, 2017 as U.S. Patent Publication No. 2017/0024546, which is incorporated by reference herein.

### TECHNICAL FIELD

The present invention relates, generally, to techniques for conducting health risk assessments and, more particularly, to an on-line platform for performing automated patient risk stratification and calculating individual wellness scores.

### BACKGROUND

The evolving U.S. health care system presents opportunities for improving population health. For example, a relatively small percentage of the population (e.g., 20-25%) consumes a disproportionate percentage of total health care costs (e.g., 70-80%), largely as a result of the treatment of chronic diseases such as diabetes, obesity, cardiovascular disease, and hypertension. Preventive measures to reverse the epidemic of chronic diseases would improve the health and life style of individual patients, improve aggregate population health, and lower overall healthcare costs.

A key component of population health involves linking clinical care with community-based prevention programs and related social services. Traditional medical education, research, and practice have focused on curing diseases for individual patients. Shifting the emphasis to embracing population-based principles can have a greater effect on long term health and wellness, particularly in the prevention of chronic disease.

At present, healthcare providers supply their eligible patients with a list of (typically non-medical) organizations offering a disease prevention program (“DPP”), relying on the patient to follow up directly with a community-based organization (“CBO”). Unfortunately, this this type of “opt-in” approach tends to result in significantly lower enrollment, in part because prevention programs offered by CBOs are not typically covered by health insurance plans.

However, when an employer offers coverage for DPPs and encourages employees to participate in such programs, the influx of a new candidate population can overwhelm enrollment resources. If a healthcare provider offers benefits which include preventive programs determining which members of the group should participate in a DPP can be cumbersome.

Systems and methods are thus needed which overcome these limitations. Various desirable features and characteristics will also become apparent from the subsequent detailed description and the appended claims, taken in conjunction with the accompanying drawings and this background section.

### BRIEF SUMMARY

The present invention provides a risk stratification system to prioritize chronic disease and lifestyle interventions for patients with multiple risk factors. The stratification system provides insight into what disease states and lifestyle interventions (as well a behavioral health and social determinants) that DPP providers should focus on for maximum impact. In addition, wellness scores generated by the stratification system can be used to prioritize patient referrals to various DPPs using a predictive analytics to select a best-fit DPP and an optimal DPP provider.

Various embodiments provide a method performed by a computer system for the automated processing of risk stratification for a patient population, calculating respective wellness scores for individual patents, assigning a risk level to each patient, and enrolling high risk patients in an appropriate DPP.

Other embodiments provide systems and methods to triage an influx of new candidates, which gives priority for preventive programs to candidates in a high health risk level over candidates in a low health risk level.

Various embodiments provide a method for performing automated triage and patient risk stratification for a population of patients, and for enrolling a high-risk patient into a disease prevention program.

Various embodiments provide systems and methods for applying predictive analytics to data from a health risk assessment, a wellness score generated by a patient risk stratification system, and individual patient preferences, to determine the best-fit DPP and the optimal DPP provider a patient. The systems and methods can be configured to enroll the patient in the best-fit DPP with the optimal DPP provider. The systems and methods may also be configured to track meaningful engagement between the patient and the optimal DPP provider.

Various other embodiments, aspects and features are described in greater detail below.

### BRIEF DESCRIPTION OF THE DRAWING FIGURES

The present disclosure will become more fully understood from the description and the accompanying drawings, wherein:

FIG. 1 is an dashboard illustrating exemplary results of a patient risk stratification over a population and wellness scores of individual patients in the population in accordance to various embodiments;

FIG. 2A is a first portion of an exemplary data sheet depicting clinical scores for the individual patients in the population of FIG. 1 in accordance with various embodiments;

FIG. 2B is a second portion of the data sheet shown FIG. 2A in accordance with various embodiments;

FIG. 3 is an exemplary data sheet depicting lifestyle scores for the individual patients in the population of FIG. 1 in accordance with various embodiments;

FIG. 4 is an exemplary data sheet depicting medication non-compliance scores for the individual patients in the population of FIG. 1 in accordance with various embodiments;

FIG. 5 is a schematic block diagram of an exemplary integrator-centric system for facilitating the provision of disease prevention programs in accordance with various embodiments;

FIG. 6 is a schematic block diagram of an exemplary system for determining a best-fit DPP and corresponding optimal DPP provider for a consumer in accordance with various embodiments;

FIG. 7 is a process flow diagram illustrating an exemplary use case involving a consumer, a payer, a DPP provider, and an integrator in accordance with various embodiments;

FIG. 8 is an exemplary wellness dashboard for an individual patient in accordance with various embodiments;

FIG. 9 is a schematic block diagram of an integrator system including an integrator computer module having a processor, a database of DPP providers, and a database of participants received from a plurality of sources in accordance with various embodiments; and

FIG. 10 is a schematic block diagram of an exemplary integrator system configured to generate a personalized precision prevention plan for three exemplary participants in accordance with various embodiments.

The drawings described herein are for illustrative purposes only of selected embodiments and not all possible implementations, and are not intended to limit the scope of any embodiment disclosed herein or any equivalents thereof.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Currently, a relatively small percentage of the U.S. population (e.g., 20-25%) consumes a disproportionate share of total health care costs (e.g., 70-80%), largely as a result of the treatment of chronic diseases such as diabetes, obesity, cardiovascular disease, and hypertension. By identifying pre-chronic disease candidates before they enter the chronic disease stage, and delivering disease prevention programs (“DPPs”) to these candidates by lower cost CBO instructors and coaches, as opposed to physicians and nurses, overall population health may be improved while reducing total healthcare costs for the population.

The present invention provides a patient risk stratification system to prioritize focus areas for chronic disease and lifestyle interventions in candidates with multiple risk factors. The stratification system provides insight into which disease states and lifestyle interventions a DPP provider should focus on for maximum impact. In addition, wellness scores generated by the stratification system can be used to prioritize candidate referrals to one or more DPPs.

In some embodiments, a patient risk stratification protocol can include processing data from a group of clinical factors, lifestyle factors, and medication compliance factors for each patient within a population, and thereafter classifying the patients into a hierarchy of risk levels and assigning a health risk status (a wellness score) to each patient. The health risk status of a patient can be used to direct the patient to a DPP designed to improve the patient's health, modify behavior, and delay or prevent the onset of a chronic disease.

Various embodiments provide methods and systems for determining an optimum DPP and/or DPP provider for which the candidate is likely to succeed, from among many DPP providers with essentially the same content, based on matching a candidate's success metrics with ideal participant profiles associated with various DPP providers and programs. The methods and systems can use predictive analytics and machine learning to pair a particular candidate with a specific one of several DPPs having analogous content, based on a prediction that the candidate will do well in the selected DPP. Identifying patient-specific risk factors allows physicians and health coaches to develop plans tailored to a patient's needs.

Various embodiments provide a “Precision Prevention Network” which imports data related to a patient's unique prevention profile to create a personalized dashboard for qualified DPPs based on patients' unique needs and preferences. The Network can create a “precision prevention plan” for a patient that predicts the best DPPs and DPP providers based on any or all of the following factors: demographics, medical information, co-morbidities, social determinants needs, program availability, patient motivation, learning environment, frequency of provider touch points with patient, language, and cultural competency. In some embodiments, a healthcare provider could transition a patient to the Network, which would then manage the patient between episodes of clinical care based on the patient's precision prevention plan.

Some embodiments provide systems and methods for providing a personalized precision prevention plan for a patient, which may be event driven by DPP milestones that monitor the patient's progress through the DPP and initiate corresponding payments to the DPP provider. The personalized precision prevention plan can be designed to enhance and encourage the patient to have meaningful engagement with the DPP provider, which increases the probability of successful program completion.

In one example, meaningful engagement contemplates the satisfaction of predetermined milestones associated with the DPP, which can then be used to trigger corresponding milestone payments from the payer to the DPP provider. For example, upon an instance of meaningful engagement, a claim may be sent to the payer and/or the DPP provider may receive a milestone payment. Meaningful engagement metrics can be tracked for a plurality of participants and a plurality of providers to improve overall system performance.

Some embodiments provide a method for performing automated triage and patient risk stratification for a patient population, and for enrolling a high-risk patient into a disease prevention program.

Performing triage on a large group of new candidates may include performing a health risk assessment for each new candidate in the group, which results in an assignment of a wellness score for each new candidate in the group, and classifying the candidates into a hierarchy of risk levels based on the wellness scores. The hierarchy of risk levels can include a high health risk level, a medium health risk, and a low health risk. The triage can prioritize the new candidates in the high health risk level for enrollment in a DPP.

In some implementations triage can include sorting data from the health risk assessment into clinical factors, lifestyle factors, and medication compliance factors, then calculating a clinical score from the clinical factors, calculating a lifestyle score from lifestyle factors, and calculating a medication non-compliance score from the medication compliance factors for each new candidate. A wellness score can be a sum (or a weighted combination) of: i) the clinical score; ii) the lifestyle score; and iii) the medication non-compliance score.

In some embodiments, the patient health risk stratification system analyzes data for the foregoing factors and correlates each of these factors independently on a predetermined scale, such as a 0-50 point scale. Using this point scale, a wellness score may comprise a total value between 0-150 points. The wellness score value increases with a patient's health risk level.

Using the wellness score having a range of 1-150 points, the health risk levels of a population can be stratified by risk as follows: high risk level is in the range of 101 to 150 points, medium risk level is in the range of 51 to 100, and a low risk level is in the range of 0 to 50. A wellness score of 150 is the highest possible score and indicates the highest health risk for a patient.

Charting the wellness score of a patient over time can demonstrate improvement, or lack thereof, of the patient in a particular area of health risk, as the patient progress through one or more DPPs. By analyzing these trends in the wellness score for a population of patients, an integrator can report metrics for improvements (or reductions) in overall population health, and can calculate or project healthcare cost savings or increases for the population, as compared to a cohort population with individuals suffering from one or more chronic diseases.

In an exemplary embodiment of the patient health risk stratification system, an individual health risk assessment guides a candidate through a series of questions, and provides data for each of the three factors (chronic disease, lifestyle risk, and medication compliance). Alternative embodiments may contemplate additional or alternate factors. Each factor may include its own set of metrics, which may be entered by the candidate through the series of questions. In some configurations of the patient health risk stratification system, data (such as, for example, lab results and/or physical measurements for the candidate) can be entered by a healthcare provider, downloaded from a healthcare plan administer, or accessed from a medical records database. As illustrated in Table 1, the candidate may input data for the metrics in three different categories of factors.

TABLE 1
Factors and Accompanying Metrics for an
Exemplary Patient Health Risk Stratification System.

| | Factors | Metrics |
|---|---|---|
| 1 | Chronic Disease | Presence and Acuity |
| 2 | Lifestyle Risk | Presence and Patient Readiness |
| 3 | Medication Compliance | Take and Fill Statistics |

According to the Centers for Disease Control and Prevention (“CDC”), the literature does not support a single uniform definition for chronic disease. However, recurrent themes include the non-self-limited nature, the association with persistent and recurring health problems, and a duration measured in months and years, as opposed to days or weeks. In general, a chronic disease is not prevented by vaccines or cured by medication.

As defined herein, a chronic disease is a medical condition which can be prevented or mitigated by modifications in behavior. In this regard, certain types of behaviors, which can be precursors for developing a chronic disease, can be assessed and entered into the patient health risk stratification system, and used as factors in determining a patient's risk for developing the chronic disease. Examples of a chronic disease, as defined herein, can include congestive heart failure (“CHF”), coronary artery disease (“CAD”), type-2 diabetes, depression, chronic obstructive pulmonary disease (“COPD”), hypertension, and hyperlipidemia.

In contrast, multiple sclerosis (“MS”), Parkinson's disease (“PD”), sickle cell disease, cystinosis, Crohn's disease, and Hashimoto thyroiditis are not classified as chronic diseases because they cannot be prevented or mitigated by changes in behavior. Accordingly, genetic disorders, autoimmune disease, and other non-preventable diseases are outside the scope of the patient health risk stratification system described herein.

Cancer, as a general term for abnormal growth of cells, is nonetheless classified as a chronic disease, although it typically cannot be prevented by modifications in behavior. However, certain types of cancer can be prevented by modifications in behavior. For example, having periodic colonoscopy examinations and modifying diet can either prevent development of or mitigate an occurrence of colon cancer. Accordingly, colon cancer is within the scope of a chronic disease, as defined herein, however, cancer, as a general term, is not within the scope of a chronic disease, as defined herein.

In general, most types of Alzheimer's disease (‘AD”) cannot be prevented by modifications in behavior. According to recent studies, behavioral risk factors have been identified which influence the likelihood of an individual developing AD. It has been reported that modifications in behavior could prevent AD in up to a third of the population at risk for AD. Thus, in some cases, AD may be within the scope of a chronic disease, as defined herein.

In addition, certain mental health diseases, such as addiction, eating disorders, and depression, can be classified as a chronic disease. If a mental health disease can be prevented or mitigated by modifications in behavior, it is within the scope of a chronic disease, as defined herein.

Other conditions contemplated by the term chronic diseases as described herein include: migraine headaches, sleep apnea, arthritis, ulcers, smoking-related lung disease, and periodontal disease.

The first factor used to characterize a chronic disease asks whether the medical condition can be prevented or mitigated by modifications in behavior. The metrics for the chronic disease are based on presence and acuity. Presence is whether or not the candidate has a condition that is a precursor to a chronic disease. Acuity is the level of severity of that condition.

Moving to the second factor, lifestyle risk includes a list of behaviors which, if continued, will increase the chances (risk) of the candidate developing one or more chronic diseases. For example, obesity, according to some studies, accounts for about 85% of the risk of developing type-2 diabetes. The metrics for lifestyle risk are presence and readiness to change. Presence is whether or not the candidate has a particular behavior. Readiness to change refers to the candidate's level of motivation to change the particular behavior.

The third factor (medication compliance) involves whether any current medications constitute a precursor to one of the chronic diseases. The metrics for medication compliance are based on the regularity that the candidate takes the medication and how often the candidate refills the medication.

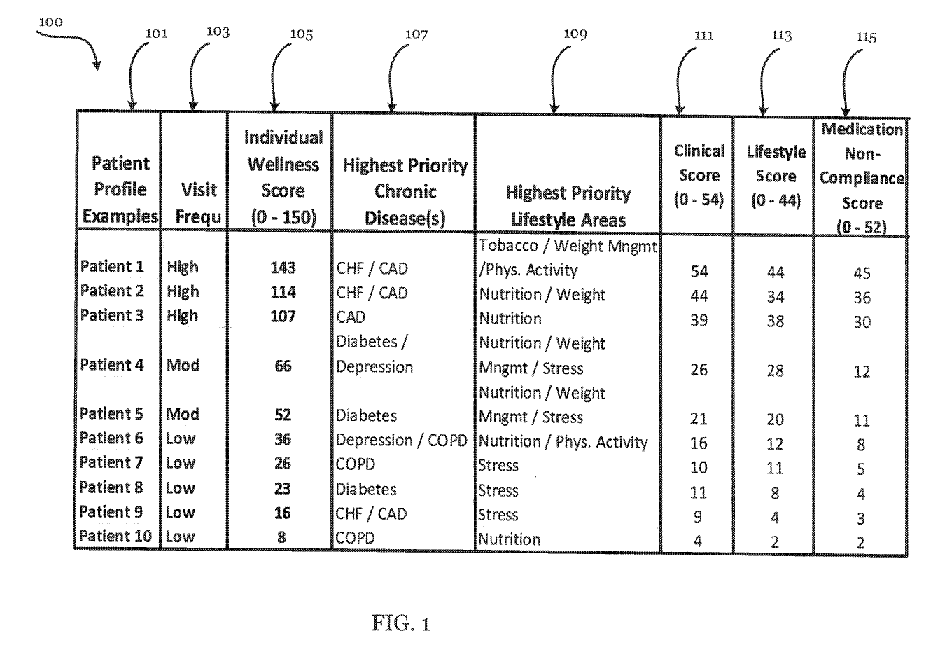

In a non-limiting example, as illustrated in FIGS. 1-4, a wellness score for an individual patient may be calculated by the patient health risk stratification system, which analyzes data from the three different factors (chronic disease, lifestyle risk, and medication compliance).

For an individual patient with multiple chronic diseases, the health risk stratification system employs a chronic disease scoring system to determine which disease, and hence, which lifestyle factors to focus on first. The chronic disease scoring system considers the likelihood of incurring a high cost chronic disease condition during a predetermined window (e.g., the next twelve months). In addition, the chronic disease scoring system factors in an assessment of the acuity of the high cost chronic disease condition.

In addition, the health risk stratification system employs a lifestyle scoring system which considers certain patient behaviors, such as whether the patient is overweight, a smoker, and/or is physically active. The lifestyle scoring system also factors in readiness of the patient to change a behavior. For example, if a patient is not ready to change a behavior, the metric for readiness to change is assigned a very low score. Conversely, if the patient is ready to change a behavior, the metric for readiness to change is assigned a very high score. The lifestyle scoring system also contemplates whether and to what degree the patient is willing to address (talk about) lifestyle behavior issues. If the patient is ready to change a behavior, the scoring system determines which of the behaviors the patient is ready to address.

The health risk stratification system also employs a medication scoring system, which involves the frequency in which the patient takes prescribed medications for a particular chronic disease. The medication scoring system also factors in whether a patient refills the prescribed medication for the particular chronic disease.

In this non-limiting example, each of the chronic disease scoring system, the lifestyle scoring system, and the medication compliance scoring system are weighted (e.g., nearly equally) when calculating the wellness score. In some configurations, the chronic disease scoring system, the lifestyle scoring system, and the medication compliance scoring system are equally weighted and all have a range from 0 to 50 points. However, in this example, the chronic disease scoring system has a range from 0 to 54 points; the lifestyle scoring system has a range from 0 to 44 points, and the medication compliance scoring system has a range from 0 to 52 points.

Referring now to FIG. 1, an exemplary dashboard 100 illustrates the results generated by a patient risk stratification system. The dashboard 100 includes the following columns: the population of individual patients 101, visit frequency 103, wellness score 105, highest priority chronic disease(s) 107, highest priority lifestyle areas 109, clinical score 111, lifestyle score 113, and medication non-compliance score 115.

The wellness score 105 is the sum of the clinical score 111, lifestyle score 113, and medication non-compliance score 115. The wellness score 105 can be used (e.g., algorithmically) to identify the duration and frequency of touch points for an individual patient. As reported in the visit frequency 103, for a wellness score in the range of 101 to 150 points, a high frequency/duration touch point schedule is recommended. For a wellness score in the range of 51 to 100, a medium frequency/duration touch point schedule is recommended, as reported in the visit frequency 103. For a wellness score in the range of 0 to 50, a low frequency/duration touch point schedule is recommended, as reported in the visit frequency 103.

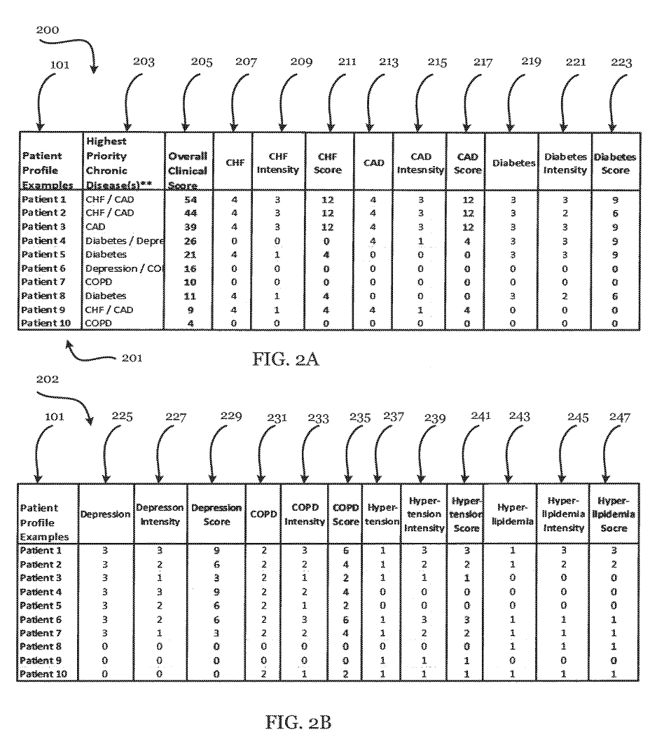

The results for the highest priority chronic disease(s) 107 and the clinical score 111 are calculated by the chronic disease scoring system, which is detailed in the datasheet of FIG. 2. The highest priority chronic disease(s) 107 can include one or more of congestive heart failure (“CHF”), coronary artery disease (“CAD”), type-2 diabetes, depression, chronic obstructive pulmonary disease (“COPD”), hypertension, and hyperlipidemia.

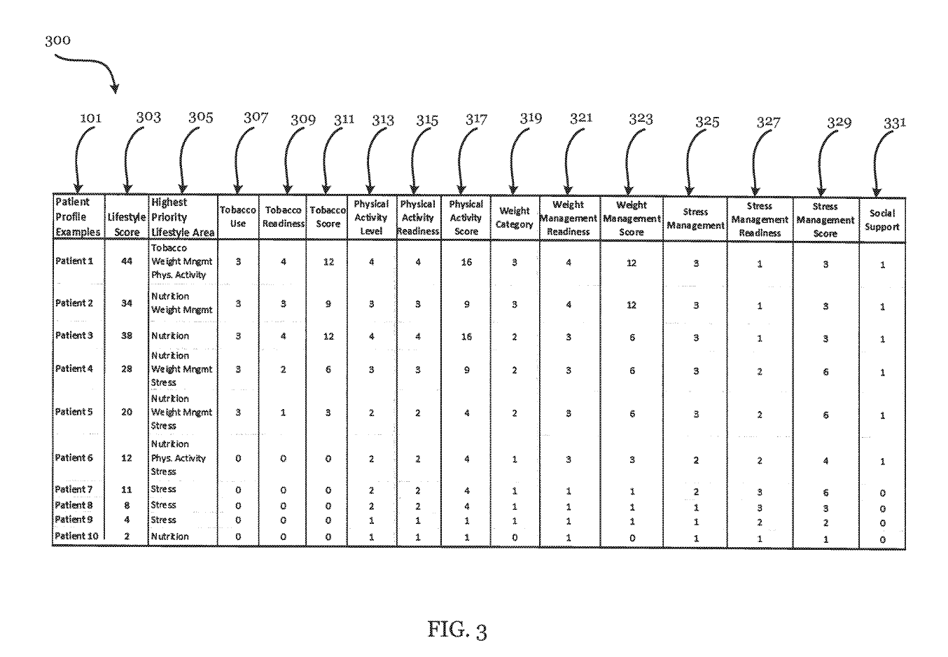

The results for the highest priority lifestyle areas 109 and the lifestyle score 113 are calculated by the lifestyle scoring system, which is detailed in the datasheet of FIG. 3. The highest priority lifestyle areas 109 can include tobacco use, physical activity level, weight management, stress management, and social support.

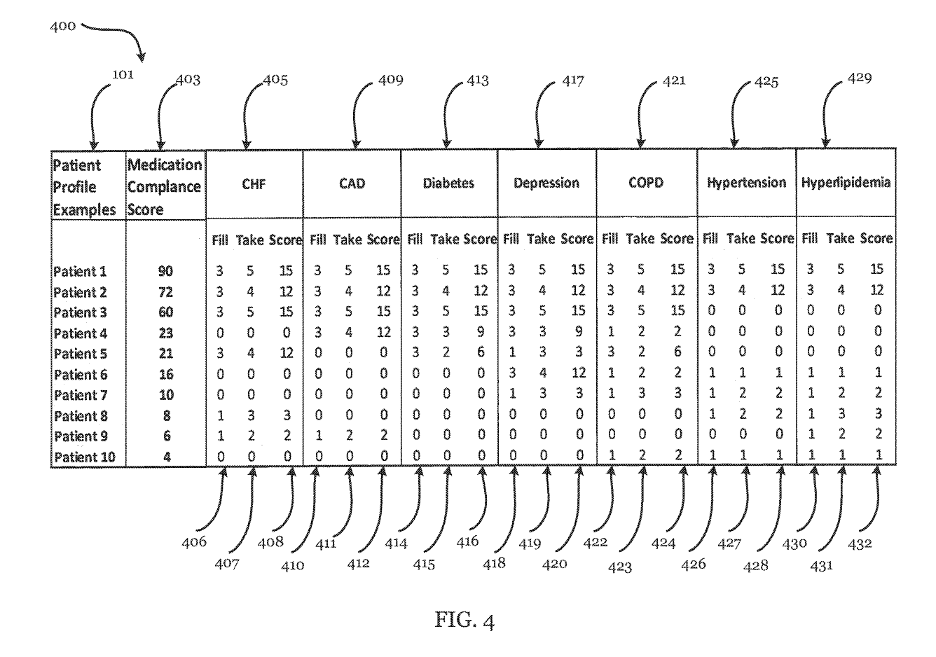

The results for medication non-compliance score are calculated by the medication compliance scoring system, which is detailed in the datasheet of FIG. 4.

FIG. 1 illustrates a patient risk stratification system configured to prioritize focus areas for chronic disease and lifestyle interventions in patients with multiple risk factors. The stratification system provides insight into those disease states 107 and lifestyle interventions 109 a DPP provider should focus on for maximum impact. In addition, wellness scores 105 generated by the stratification system can be used to prioritize patient referrals to one or more DPPs.

The dashboard 100 can also be used to assist the integrator in determining a patient's best-fit DPP based on the optimal frequency and duration of touch points between clinical care instances to maximize compliance with the patient's care plan between doctor visits.

Turning to FIG. 2, an exemplary datasheet 200 for calculating a clinical score is broken into 2 parts due the length of the columns of data. In particular, FIG. 2A is a first portion 201 of the exemplary data sheet for calculating a clinical score, which connects to FIG. 2B, a second portion 202 of the exemplary data sheet for calculating a clinical score. The overall clinical score 205 is calculated using the data in both the first portion 201 and the second portion 202 of the exemplary data sheet 200.

The list of chronic diseases on datasheet 200 includes congestive heart failure (“CHF”), coronary artery disease (“CAD”), type-2 diabetes, depression, chronic obstructive pulmonary disease (“COPD”), hypertension, and hyperlipidemia.

The datasheet 200 includes the following columns: the population of patients 101, highest priority chronic disease(s) 203, overall clinical score 205, CHF DZ points 207, CHF intensity 209, CHF score 211, CAD DZ points 213, CAD intensity 215, CAD score 217, diabetes DZ points 219, diabetes intensity 221, diabetes score 223, depression DZ points 225, depression intensity 227, depression score 229, COPD DZ points 231, COPD intensity 233, COPD score 235, hypertension DZ points 237, hypertension intensity 239, hypertension score 241, hyperlipidemia DZ points 243, hyperlipidemia intensity 245, and hyperlipidemia score 247.

An individual chronic disease score is the product of a first value associated with the presence of the chronic disease, coupled with (e.g., multiplied by) a second value associated with the acuity of the chronic disease.

With regard to the value associated with the presence of a chronic disease, each chronic disease is assigned disease points, (labeled as DZ points in Table 2) which in the illustrated example range from 1 to 4. As listed in Table 2, the disease points are determined for each chronic disease based the severity, the cost of, and the likelihood of hospitalization for the patient in next 12 months. If a patient does not have a particular chronic disease, the assigned value for the presence of the disease is zero.

For the acuity of a chronic disease, an intensity level is assigned based on clinical results, which in the illustrated example ranges from 1 to 3. The intensity level for each chronic disease is described in Table 3. If a patient does not have a particular chronic disease, the intensity level is zero.

TABLE 2
Disease Point System for Chronic Diseases.

| Chronic | |
|---|---|
| Disease | DZ Points (1-4) |
| CHF | 4 |
| CAD | 4 |
| Diabetes | 3 |
| Depression | 3 |
| COPD | 2 |
| Hypertension | 1 |
| Hyperlipidemia | 1 |

TABLE 3
Intensity Level Point System for Chronic Diseases.

| | | | Intensity |
|---|---|---|---|
| CHF | | | Points |
| INTENSITY LEVEL = 1 if patient answers “NO” to | | | 1 |
| question (1): Do you wake up at night to catch your | | | |
| breath?, AND patient answers “NO” to question (2): Do | | | |
| you need more than two pillows under your head at night | | | |
| to breathe comfortably?, AND patient answers “NO” to | | | |
| question (3): Do you get short of breath walking around | | | |
| the house? | | | |
| INTENSITY LEVEL = 2 if patient answers “NO” to | | | 2 |
| question (1): Do you wake up at night to catch your | | | |
| breath?, AND patient answers “NO” to question (2): Do | | | |
| you need more than two pillows under your head at night | | | |
| to breathe comfortably?, AND patient answers “YES” to | | | |
| question (3): Do you get short of breath walking around | | | |
| the house? | | | |
| INTENSITY LEVEL = 3 if patient answers “YES” to | | | 3 |
| question (1): Do you wake up at night to catch your | | | |
| breath?, AND patient answers “YES” to question (2): Do | | | |
| you need more than two pillows under your head at night | | | |
| to breathe comfortably?, AND patient answers “YES” to | | | |
| question (3): Do you get short of breath walking around | | | |
| the house? | | | |
| | | | Intensity |
| CAD | | | Points |
| INTENSITY LEVEL = 1 if patient has 1 of the following | | | 1 |
| 7 cardiac risk factors | | | |
| INTENSITY LEVEL = 2 if patient has 2 of the | | | 2 |
| following 7 cardiac risk factors | | | |
| INTENSITY LEVEL = 3 if patient has 3 or more of the | | | 3 |
| following 7 cardiac risk factors | | | |
| (1)Hypertension | | | |
| (2)Depression | | | |
| (3)LDL > 130 mg/dL | | | |
| (4)Family History of Heart Disease | | | |
| (5)Obesity | | | |
| (6)Tobacco Use | | | |
| (7)Diabetes | | | |
| Diabetes | Fasting Blood | Non-fasting | Intensity |
| | Glucoe | Blood Glucose | Points |
| | ≥120 | ≥194 | 1 |
| | ≤125 mg/dL | ≤199 mg/dL | |
| | ≥126 | ≥200 | 2 |
| | ≤166 mg/dL | ≤240 mg/dL | |
| | ≥167 mg/dL | ≥241 | 3 |
| | | | Intensity |
| COPD | | | Points |
| INTENSITY LEVEL = 1 if Pulse Oximeter | | | 1 |
| measurement = 95% Oxygen or above OR patient reports | | | |
| no shortness of breath | | | |
| INTENSITY LEVEL = 2 if Pulse Oximeter | | | 2 |
| measurement = 90-95% Oxygen OR patient reports | | | |
| shortness of breath with moderate exertion (one flight of | | | |
| stairs) | | | |
| INTENSITY LEVEL = 3 if Pulse Oximeter | | | 3 |
| measurement < 90% Oxygen OR patient reports | | | |
| shortness of breath with mild exertion (walking around | | | |
| house) | | | |
| | | | Intensity |
| Hypertension | Systolic | Diastolic | Points |
| Higher intensity | ≥140 ≤149 | ≥90 ≤94 | 1 |
| score if either | ≥150 ≤159 | ≥95 ≤99 | 2 |
| systolic OR | ≥160 | ≥100 | 3 |
| diastolic value | | | |
| falls in the higher | | | |
| range | | | |

| | | Intensity |
|---|---|---|
| Hyperlipidemia | LDL Level | Points |
| | ≥130 | 1 |
| | ≤159 mg/dL | |
| | ≥160 | 2 |
| | ≤189 mg/dL | |
| | ≥190 mg/dL | 3 |

| | Intensity |
|---|---|
| Depression | Points |
| INTENSITY LEVEL = 1 if patient “feels blue” 1-3 days | 1 |
| out of 7 days of the week | |
| INTENSITY LEVEL = 2 if patient “feels blue” 4-5 | 2 |
| days out of 7 days of the week | |
| INTENSITY LEVEL = 3 if patient “feels blue” 6 or 7 | 3 |
| days out of 7 days of the week | |

Biometrics can be used to input the clinical results to determine the intensity level of a chronic disease. In some embodiments the intensity level of a chronic disease can be entered by a healthcare provider, downloaded from a healthcare plan administer, or accessed from a medical records database.

The overall clinical score 205 is calculated by the chronic disease scoring system. The overall clinical score 205 is the sum of the chronic disease scores. That is, the overall clinical score 205 is the sum of the CHF score 211, the CAD score 217, the diabetes score 223, the depression score 229, the COPD score 235, the hypertension score 241, and the hyperlipidemia score 247. The overall clinical score 205 has a range from 0 to 54 points.

A chronic disease score is the product of presence of the chronic disease multiplied by the acuity of the chronic disease. The CHF score 211 is the product of the CHF DZ points 207 multiplied by the CHF intensity 209. CAD score 217 is the product of the CAD DZ points 213 multiplied by the CAD intensity 215. The diabetes score 223 is the product of the diabetes DZ points 219 multiplied by the diabetes intensity 221. The depression score 229 is the product of the depression DZ points 225 multiplied by the depression intensity 227. The COPD intensity 233 is the product of the COPD DZ points 231 multiplied by the COPD score 235. The hypertension score 241 is the product of the hypertension DZ points 237 multiplied by the hypertension intensity 239. The hyperlipidemia score 247 is the product of the hyperlipidemia DZ points 243 multiplied by the hyperlipidemia intensity 245.

The highest priority chronic disease(s) 203 is determined by the highest score for a chronic disease using the combination of disease presence, disease intensity, self-management compliance, and readiness to change. If the highest score is similar for two or more chronic diseases, the highest priority chronic disease(s) 203 can contain more than one chronic disease.

Each patient listed in the population of individual patients 101 has an individual listing of chronic diseases in the highest priority chronic disease(s) 203, and an independent overall clinical score 205 which is calculated using the patient's data.

The results in the highest priority chronic disease(s) 203 automatically populate the fields in the highest priority chronic disease(s) 107 in the dashboard 100 illustrated in FIG. 1. The results in the overall clinical score 205 automatically populate the fields in the overall clinical score 111 in the dashboard 100 illustrated in FIG. 1.

FIG. 3 is an illustration of an exemplary datasheet 300 for calculating a lifestyle score. The datasheet 300 includes the following columns: lifestyle score 303, highest priority lifestyle area 305, tobacco use 307, tobacco readiness 309, tobacco score 311, physical activity level 313, physical activity intensity 315, physical activity score 317, weight category 319, weight management readiness 321, weight management score 323, stress management 325, stress management readiness 327, stress management score 329, and social support 331.

A lifestyle score is the product (or other combination) of a first value representing the presence of a lifestyle behavior, and a second value representing the readiness to change the lifestyle behavior. For the presence of a lifestyle behavior, each lifestyle behavior is assigned points, which may range from 1 to 4. As listed in Table 4, the points are determined for each lifestyle behavior based the severity of the behavior.

TABLE 4
Point System for Lifestyle Behavior.

| | Points |
|---|---|
| Tobacco Use | |
| Current Tobacco User | 3 |
| Non-tobacco user | 0 |
| Physical Activity | |
| Level | |
| Sedentary | 4 |
| Low Active | 3 |
| Active | 2 |
| Very Active | 1 |
| Weight Category | |
| Underweight | 2 |
| Normal | 0 |
| Overweight | 1 |
| Obese | 2 |
| Morbidly Obese | 3 |
| Stress | |
| Low Stress | 1 |
| Moderate Stress | 2 |
| High Stress | 3 |
| Social Support | |
| Yes | 0 |
| No | 1 |

For readiness to change the lifestyle behavior, a readiness score is assigned, based on motivation, which may range from 1 to 4. A high readiness score indicates the patient is ready to change a particular behavior. The readiness score is described in Table 5. If a patient has a zero for a lifestyle behavior, the readiness score is zero.

TABLE 5
Point System for Readiness to Change a
Lifestyle Behavior.

| | Readiness |
|---|---|
| Readiness Scores | (1-4) |
| Relapse | 4 |
| Not ready to change | 1 |
| Ready (contemplation) | 4 |
| Preparation | 3 |
| Action | 2 |
| Action after relapse | 2 |
| Maintenance | 1 |

The lifestyle score 303 is calculated by the lifestyle scoring system. The lifestyle score 303 is the sum of the lifetime scores.

Accordingly, the lifestyle score 303 is the sum of the tobacco score 311, the physical activity score 317, the weight management score 323, the stress management score 329, and the social support score 331. The lifestyle score 303 has a range from 0 to 44 points.

A lifestyle score is the product of the presence of the lifestyle behavior multiplied by the readiness to change the lifestyle behavior. The tobacco score 311 is the product of the tobacco use 307 multiplied by the tobacco readiness 309. The physical activity score 327 is the product of the physical activity level 313 multiplied by the physical activity readiness 315. The weight management score 323 is the product of the weight category 319 multiplied by the weight management readiness 321. The stress management score 329 is the product of the stress management 325 multiplied by the stress management readiness 329.

The highest priority lifestyle area 305 is determined by the highest score for a lifestyle behavior using the combination of lifestyle behavior presence, and readiness to change. If the highest score is similar for two or more lifestyle behaviors, the highest priority lifestyle area 305 can contain more than one lifestyle behavior. The highest priority lifestyle areas 305 can include one or more of tobacco use, physical activity level, weight management, stress management, and social support.

Each patient listed in the population of individual patients 101 has an individual listing of lifestyle behaviors in the highest priority lifestyle area 305, and an independent lifestyle score 303, which is calculated using the patient's data.

The results in the highest priority lifestyle area 305 automatically populate the fields in the highest priority lifestyle area 109 in the dashboard 100 illustrated in FIG. 1. The results in the lifestyle score 303 automatically populate the fields in the lifestyle score 113 in the dashboard 100 illustrated in FIG. 1.

FIG. 4 illustrates an exemplary data sheet 400 for calculating a medication non-compliance score. The datasheet 400 includes the following columns: the population of patients 101, medication compliance score 403, CHF (medication) 405 having fill 406, take 407, and score 408, CAD (medication) 409 having fill 410, take 411, and score 412, diabetes (medication) 413 having fill 414, take 415, and score 416, depression (medication) 417 having fill 418, take 419, and score 420, COPD (medication) 421 having fill 422, take 423, and score 424, hypertension (medication) 425 having fill 426, take 427, and score 428, and hyperlipidemia (medication) 429 having fill 430, take 431, and score 432.

A medication score for each condition is the product (or other combination) of a first value related to filling a prescription, and a second value related to whether the medication is taken as prescribed. With regard to the filling of a prescription, each action may be assigned points ranging from 1 to 3. With regard to taking the medication as prescribed, each action may be assigned points ranging from 1 to 5. As listed in Table 6, the points are determined for filling and taking medication based on an action.

TABLE 6
Point System for Medication Compliance.

| Medication | | |
|---|---|---|
| Compliance Factors | Action | Points |
| Regularly fills prescription | Yes | 1 |
| | No | 3 |
| Takes prescription as | Always | 1 |
| prescribed | Frequently | 2 |
| | Usually | 3 |
| | Rarely | 4 |
| | Never | 5 |
| Has no prescriptions | None | 0 |

The medication compliance score 403 is calculated by the medication compliance scoring system. The medication compliance score 403 may comprise one half of the sum of the medication compliance scores. That is, the medication compliance score 403 may be one half of the sum of the CHF score 408, the CAD score 412, the diabetes score 416, the COPD score 424, the hypertension score 428, and the hyperlipidemia score 432. If appropriate, the medication compliance score 403 may be rounded down to a whole number. The medication compliance score 403 has an exemplary range from 0 to 52 points.

A medication compliance score may be determined for each condition by combining (e.g., multiplying) a first value relating to filling a prescription with a second value relating to taking the medication as prescribed. In the illustrated embodiment, the CHF score 408 is a product of the CHF fill 406 multiplied by the CHF take 407. The CAD score 412 is a product of the CAD fill 410 multiplied by the CAD take 411. The diabetes score 416 is a product of the diabetes fill 414 multiplied by the diabetes take 415. The COPD score 424 is the product of the COPD fill 422 multiplied by the COPD take 423. The hypertension score 428 is the product of the hypertension fill 426 multiplied by the hypertension take 427. The hyperlipidemia score 432 is the product of the hyperlipidemia fill 430 multiplied by the hyperlipidemia take 431.

The results in the medication compliance score 403 automatically populate the fields in the medication non-compliance score 115 in the dashboard 10o illustrated in FIG. 1.

The systems and methods of patient health risk stratification are not limited to the factors discussed in FIGS. 1-4. Demographic and socioeconomic factors can be added to the patient health risk stratification. The demographic and socioeconomic factors can be equally or differently weighted relative to the other factors used in the patient health risk stratification (in a range of 0-50 points in this example).

In addition, behavioral health risks can be added as a factor in the patient health risk stratification. The behavioral health factors can be weighed equally or differently relative to the patient health risk stratification (in a range of 0-50 points in this example). If the behavioral health factors are used, depression may be added to these factors and removed from the chronic disease datasheet. The behavioral health risks factors can be determined by charting the presence of a behavioral disease and the severity of the behavioral disease (similar to the chronic disease calculation).

If both the demographic and socioeconomic factors and the behavioral health factors are used in the patient health risk stratification, an exemplary wellness score may range from 0-250 points. In addition, the risk level hierarchy may be as follows: high risk level is in the range of 166 to 250 points, medium risk level is in the range of 81 to 165, and a low risk level is in the range of 0 to 80.

Various embodiments of the present invention relate to systems and methods for linking a candidate (also referred to herein as a patient, a participant, consumer, or a user) with non-medical DPP providers to provide DPPs based on the candidate's input into a risk assessment and into a survey of program preferences. The present invention further contemplates systems and methods to facilitate determining a best-fit DPP for a particular candidate based on the candidate's input.

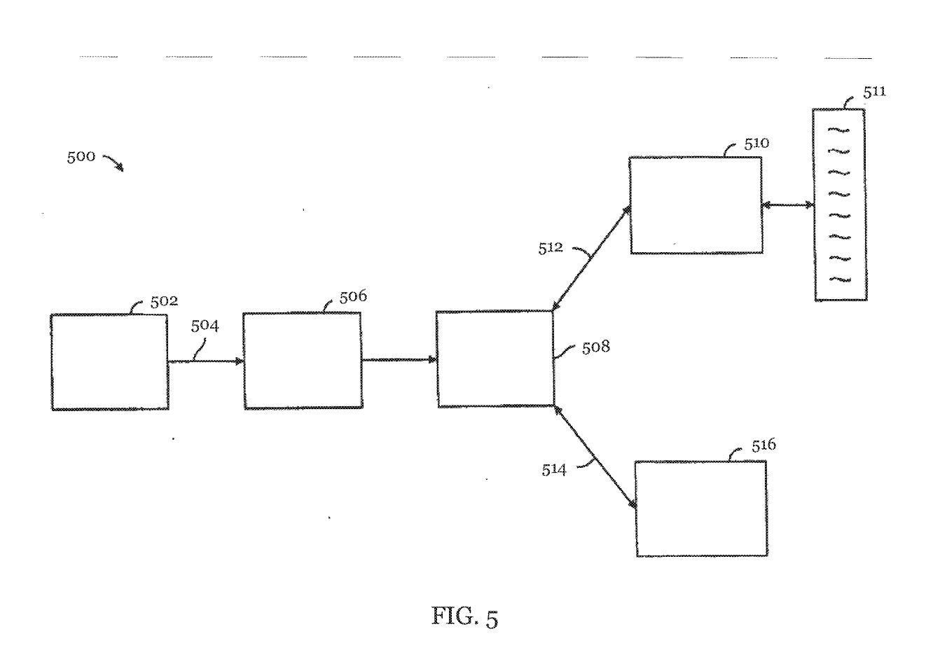

More particularly and referring now to FIG. 5, an exemplary integrator-centric system 500 for delivering at least one DPP includes a clinical provider 502 configured to refer 504 a candidate 506 to an integrator 508. The clinical provider 502 includes, but is not limited to a doctor or a hospital. The refer 504 can be in the form of a professional referral or a prescription. The candidate 506 can include, but is not limited to, a patient, a client of a medical service, a program participant, a consumer, and/or a user.

The candidate 506 enters data responsive to a health and lifestyle survey. These data are analyzed by a patient health risk stratification system, which is configured to recognize more than one chronic disease and configured to determine the highest priority chronic diseases for the candidate 506. A list of the highest priority chronic diseases is used match a corresponding DPP for each chronic disease on the list.

However, the process of matching a candidate 506 to a best-fit DPP 510 involves determining whether the candidate 106 is eligible for one or more DPPs 510 based on objective criteria, as defined by the candidate's health plan (e.g., payer 516). For example, a candidate with a wellness score in the low risk level may not be eligible for benefits covering a DPP.

If the candidate 506 is eligible for any of the corresponding DPP for each chronic disease on the list, the candidate 506 inputs data responsive to a personal profile survey.

The integrator 508 accesses a database 511 of DPP providers and recommends a “best fit” DPP 510 based on, for example, correlation between the personal profile entered by the candidate 506 and one of a plurality of ideal participant profiles, each associated with a DPP provider. The integrator 508 enrolls the candidate 506 into the best-fit DPP 510.

The integrator 508 can prioritize the candidate 506 for the “best-fit” DPP using a wellness score. In an example, the wellness score is generated by the patient health risk stratification system described in FIGS. 1-4.

The integrator 508 monitors 512 the candidate's compliance with the DPP 510, and processes a claim for payment 514 from a payer 516. The payer 516 can be an insurance company, Medicaid, Medicare, a health system, or health plan administrator. The integrator 508 may be configured to process a claim for payment by performing one of more of the steps of: submitting a claim to the payer 514, receiving approval for the claim from the payer 116, invoicing the payer 516, and receiving the payment for the claim from the payer 516. The integrator 508 can send the initial claim to the payer 516 upon enrollment of the candidate 506 in the DPP 510.

Charting the wellness score of the candidate 506 over time can demonstrate improvement, or lack thereof, of the candidate 506 in a particular area of health risk, as the candidate 506 progresses through one or more DPPs.

In accordance with various embodiments, the system 500 programmatically determines an ideal personality profile based on the outcomes of the most successful participants for each DPP provider. Each DPP provider can have one or more vehicles for delivering a DPP 510. For example, a DPP provider can offer a qualifying DPP 510 in a group session and alternatively, can offer another qualifying DPP 510 using a virtual interface.

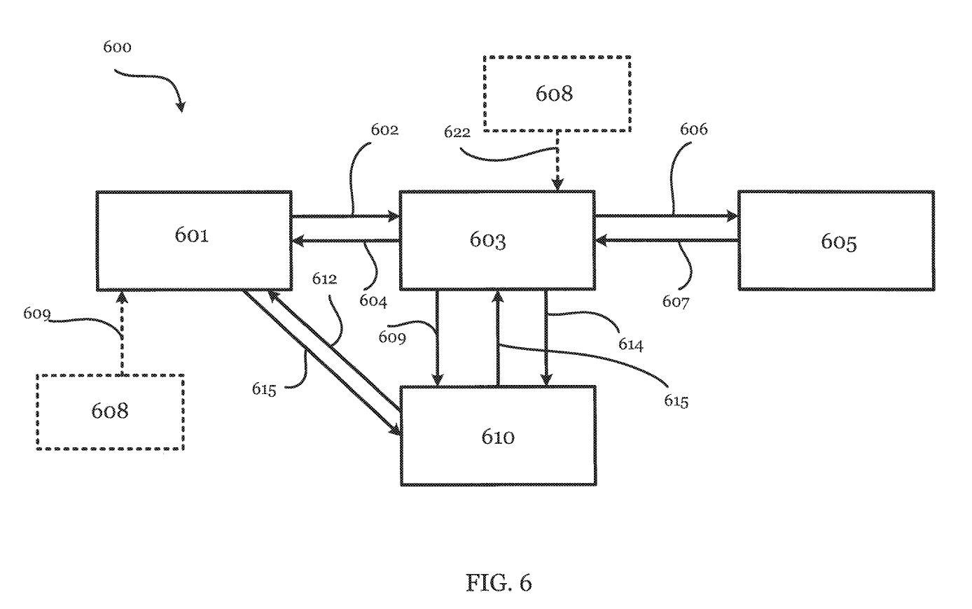

With reference to FIG. 6, an exemplary system 600 for implementing a three sided marketplace metaphor includes an integrator 603, a consumer 601, a payer 605, and a DPP provider (such as a CBO) 610. In this regard, each of the consumer 601, the DPP provider 610, and the payer 605 interface with the integrator 203 at different events from enrolling the consumer in an appropriate DPP, and thereafter through program monitoring, completion, and payment.

The consumer 601 initiates a sequence of events (collectively referred to herein as the transaction) by making contact with the integrator 603 for enrollment in a DPP.

The consumer 601 inputs data 602 responsive to a health risk assessment. These data are analyzed by a patient health risk stratification system, which is configured to recognize more than one chronic disease and is configured to determine the highest priority chronic diseases for the consumer 601 by using algorithms. The integrator 603 generates a list of the highest priority chronic diseases, which is used to identify a corresponding DPP for each chronic disease on the list. Exemplary FIG. 1 includes a listing of the highest priority chronic disease(s) 107 for each patient profile 101.

A wellness score can be used to classify a priority (based on a health risk level) of the consumer 601 for one or more DPPs. The wellness score value increases with increasing health risk of the consumer 601. For example, the wellness score can have a range of 1-150 points and the health risk levels can be as follows: high risk level is in the range of 101 to 150 points, medium risk level is in the range of 51 to 100, and a low risk level is in the range of 0 to 50. Exemplary FIG. 1 includes a wellness score 105 for each patient profile 101. If the consumer 601 has been classified in at a high risk level, the system increases the priority of placing the consumer 601 into the needed DPPs.

If the consumer 601 is eligible for a corresponding DPP for each chronic disease on the list, the consumer 601 inputs data responsive to a personal preference survey. The integrator's computer system determines the best-fit DPP provider 610 by using analytics to compare the personal preferences of the consumer 601 to ideal personal preferences for each of a plurality of DPP providers. In addition, the consumer 601 can enter a location, which is the location that the consumer 601 will be before traveling to the DPP provider 610, for example, home, work, school, the gym, or the office. The location of the consumer 201 is compared to a location of each of the plurality of DPP providers. Based on this analysis of personal preferences and locations, a best-fit DPP provider 610 is identified. The integrator 603 enrolls the consumer 601 in the best-fit DPP with the best-fit DPP provider 610. Notice of enrollment 204 is sent to the consumer 601. A second notice of enrollment 609 is sent to the best-fit DPP provider 610.

Upon enrollment, the integrator 603 sends a claim 606 to payer 605. The payer 605 sends an approval or payment 207 to integrator 203. The consumer 601 participates 613 in the DPP and the DPP provider 610 delivers the DPP content 612 to the consumer 201. The DPP provider 610 reports 614 progress and other data to the integrator 603. Once the integrator 603 identifies that the consumer 601 has satisfied a milestone or completed the DPP, the integrator 603 sends a payment 615 to the DPP provider 610, which ends the transaction or advances the transaction to the next milestone.

Some embodiments include an option of a traditional, clinical healthcare provider 608 (such as a physician) providing the consumer 601 a prescription 609 (or a professional referral) for a DPP, which may include instructions for contacting the integrator 603. Some embodiments include an option of the integrator 603 providing a report 620 to the healthcare provider 608.

In some cases, a candidate consumer may be in need of more than one DPP. For example, candidate consumer may be in need DPPs for one or more of congestive heart failure (“CHF”), coronary artery disease (“CAD”), type-2 diabetes, depression, chronic obstructive pulmonary disease (“COPD”), hypertension, and hyperlipidemia. The integrator system can include a patient health risk stratification system configured to recognize more than one chronic disease and determine the highest priority chronic diseases for a candidate consumer.

In one embodiment, a consumer's health risk assessment is driven by machine learning to analyze previous answers and determine if additional question strings need to be added to the assessment, which evaluate the candidate consumer for other disease conditions.

The integrator system can be configured use a list of the highest priority chronic diseases to identify a corresponding DPP for each chronic disease on the list. The integrator system can be configured to administer multiple DPPs for a consumer. Depending of the group of multiple DPPs, the timing of the consumer taking each DPP may be simultaneously (all at once), sequentially, overlapping, or some together and others later in time. The integrator system can be configured to administer the scheduling and coordinating any timing structure for a consumer taking a group of multiple DPPs.

In some embodiments, the integrator system can include a Precision Prevention Network, which is configured to import from the health risk stratification system to create a personalized dashboard for not only qualified DPPs, but also the type and delivery intervention method of the DPPs based on the consumer's unique needs and preferences. The Network can create a precision prevention plan for a consumer that predicts the best DPPs and DPP providers based on any or all of the following factors: use a list of the highest priority chronic diseases, a list of a wellness number, demographics, medical information, co-morbidities, social determinants needs, DPP availability, prioritization for a DPP, patient motivation, learning environment, frequency of DPP provider touch points with the consumer, language, and cultural competency. In some embodiments, a healthcare provider transitions the consumer to the Network, which is configured to manage the consumer between episodes of multiple DPPs based on the consumer's precision prevention plan.

In some embodiments, a method of patient risk stratification can include processing a group of clinical factors, lifestyle factors, and medication compliance factors, for each patient of a population; classifying the population of patients into a hierarchy of risk levels; and assigning a health risk status (a wellness score) to each patient of the population, which is based on the group of factors. The method can include accessing the health risk status of a patient and directing the patient to a DPP, which is designed to improve the patient's health, to establish changes in behavior, and to prevent development of a chronic disease by the patient.

The method can include accessing the health risk status of a patient; accessing medical records of the patient; combining the health risk status and the medical record with demographic and socioeconomic characteristics for the patient; and creating a comprehensive patient profile. The method can include combining the health risk status and the medical record; and determining a list of chronic diseases that the patient may develop in the next year. The method can include assigning the patient to a DPP corresponding to one of the chronic diseases on the list. The method can include mapping the comprehensive patient profile over a group of DPP providers qualified to deliver the DPP; and determining the optimal DPP provider for the patient. The method can include enrolling the patient in the DPP with the optimal DPP provider.

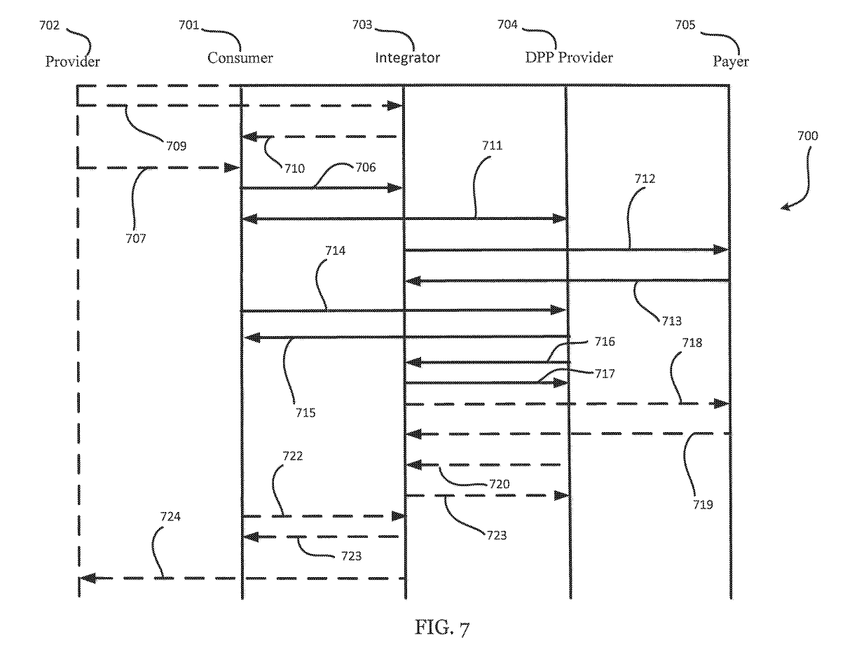

FIG. 7 is a process flow diagram 700 illustrating an exemplary use case involving a consumer 701, an integrator 703, a DPP provider 704, a payer 705, and optionally a healthcare provider 702. All of these parties have been described in detail in various portions of this application.

Some embodiments include an option of the healthcare provider 702, (for example, a doctor, a clinic, a hospital or a health care system) referring a consumer 701 to the integrator 703 (Step 709), whereupon the integrator 703 contacts the consumer 701 and invites the consumer 701 to log into the integrator's system (e.g., on-line portal) and to find a DPP that is a best-fit for the consumer 701 (Step 710).

Some embodiments include an option of the healthcare provider 702 providing the consumer 701 with a prescription or other instruction to attend a DPP, which may include instructions for contacting the integrator 703 (Step 707).

In an embodiment, the consumer 701 contacts the integrator 702 through a portal and provides various data points, such as, for example, the DPP program desired, personal information, such as, name, address, zip code, associated payer 705 information, and the referral or prescription, which is used to set up an account in the integrator's system (Step 706).

The integrator 703 walks the consumer 701 through a survey to create a health risk assessment. In some embodiments, the responses can be entered into a health risk stratification system, which can generate a list of the highest priority chronic disease(s) and a list of the highest priority lifestyle areas. The list of the highest priority chronic diseases can be used by the system to match a corresponding DPP for each chronic disease on the list. The list of the highest priority lifestyle areas can be entered as part of the matrix of personal preferences. A wellness score can be generated for the consumer 701 by the patient health risk stratification system, for example, as described in FIGS. 1-4. The wellness score increases with increased health risk of the consumer 701. A high wellness score (high risk) can signal the system to prioritize the consumer 701 for DPPs over other consumers with low wellness scores (low risk).

The integrator 703 continues to walk the consumer 701 through matrix of personal preferences, which can include preferred modes of content delivery, location, time/days for sessions, group dynamics, virtual options, and the consumer's level of motivation to complete the DPP. The matrix of personal preferences generates a personality profile, preferably with a location. The personality profile can be mapped against a plurality of ideal personality profiles associated with the DPP providers 704.

Using the results of the personality profile analysis and the consumer's location (e.g., home or work address), the integrator 703 determines which DPP provider 704 is the best-fit DPP provider 704 for the consumer 701. The integrator 703 enrolls the consumer 701 in the desired DPP with the best-fit DPP provider 704. The integrator 703 sends notice of the enrollment (which can include a DPP class schedule and any other information about the DPP, such as dress code, dietary restrictions, or required monitoring systems to both the consumer 701 and the DPP provider 704 (Step 711).

However, if the consumer 701 is already affiliated with a particular health plan from the payer 705, the integrator 703 may permit the payer or the consumer to designate a preferred DPP provider 704, as the best-fit DPP provider 704 for one or more of the DPPs for which the consumer is eligible.

Upon the enrollment of the consumer 701, the integrator 703 prepares and sends a claim to the payer 705 (Step 712). The integrator 703 receives an approval of the claim from the payer 705, which may include a partial payment claim (Step 713).

The consumer 701 participates in the DPP (Step 714). The DPP provider 704 provides the resources and delivers the content of the DPP to the consumer 701 (Step 715). As the consumer 701 progresses through the DPP, the DPP provider 704 updates the consumer's record and progress within a shared database maintained by the integrator 703 (Step 716).

In some embodiments, the integrator 703 may provide an interactive software tool for use by the DPP provider 704 to facilitate the integration process, for example, by allowing the DPP provider 304 to enter consumer data (e.g., attendance, body weight, and the like) directly into consumer's records maintained by, on behalf of, or at the direction of the integrator 703. In an embodiment, such an interactive software tool may include the Solera™ technology platform program available from Solera™ Health, Inc. located in Phoenix, Ariz.

Charting the wellness score of the consumer 701 over time can demonstrate improvement, or lack thereof, of the consumer 701 in a particular area of health risk, as the consumer 701 progresses through one or more DPPs. One of the goals of the DPP provider 704 is to lower the wellness score of the consumer 701, which indicates a reduction in the health risk of the consumer 701.

By analyzing these trends in the wellness score for a population of consumers, an integrator 703 can report metrics for the improvements in overall health of the population, and can calculate healthcare cost savings for the population of consumers, as compared to a cohort population with individuals suffering from one or more chronic disease.

Upon completion of the DPP or, alternatively, at various predetermined milestones, the integrator 703 makes a partial or full payment to the DPP provider 704 (Step 717).

In some embodiments, if multiple milestones are required for program completion, the system 700 can be setup to prepare and send one or more interim or supplemental claim to the payer 705 upon completion of each milestone (Step 718). The integrator 703 receives an approval of the interim or supplemental claim, which may include a partial payment (Step 719). The DPP provider 704 continues to update the consumer's record and progress within the shared database maintained by the integrator 703 (Step 720). Upon completion of the DPP or, alternatively, at the next predetermined milestone, the integrator 703 makes an additional partial or final payment to the DPP provider 704 (Step 721). The optional Steps 718 thru 721 can be repeated multiple times, as determined by the number of milestones that are in a particular DPP.

In some embodiments, the integrator 703 may send the consumer 701 a survey or otherwise solicit feedback at certain times during the DPP (Step 723). The consumer 701 completes the survey and the survey results are stored by the integrator 703. The survey can be directed to the quality and efficiency of the DPP and/or the DPP provider. Using machine learning capabilities of the integrator system, the results of a group of surveys can be analyzed to modify the ideal personality profile and/or other metrics for the DPP provider 704. In addition, the results of a group of surveys can be used to rank the DPP provider 704 among a population of DPP providers in a network.

In another embodiment, the consumer 701 can track data and milestones by accessing a dashboard provided by the integrator 703 (Step 722). The integrator 703 continually updates the data and populates the fields in the dashboard for viewing by the consumer 701 (Step 723).

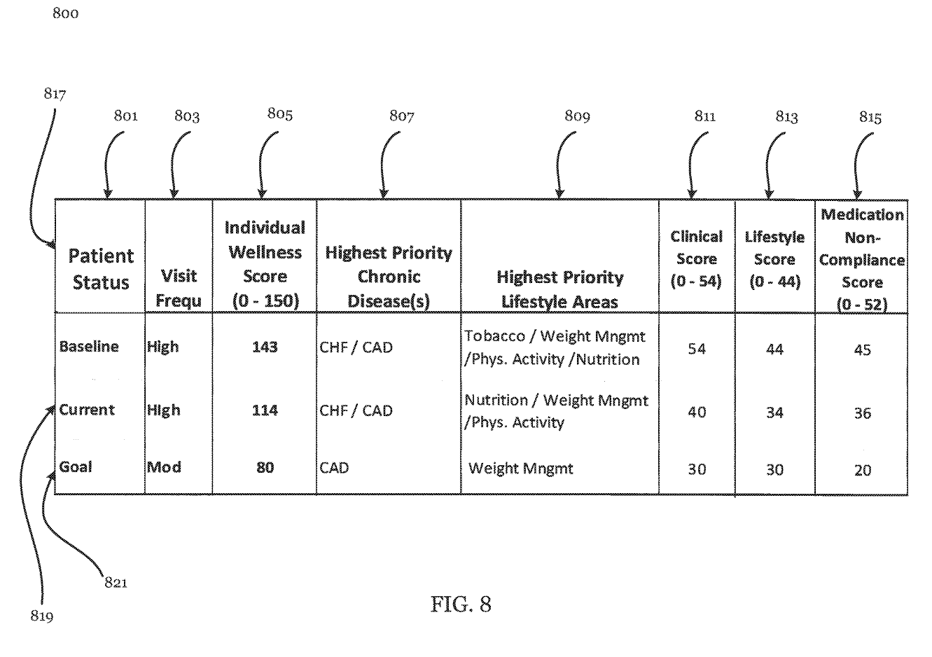

An example of a consumer dashboard Boo is illustrated in FIG. 8. The consumer dashboard Boo may include, inter alia, one or more of the following columns: patient profile 801, visit frequency 803, wellness score 805, highest priority chronic disease(s) 807, highest priority lifestyle areas 809, clinical score 811, lifestyle score 813, and medication non-compliance score 815.

The consumer dashboard Boo may also include one or more of the following rows: baseline row 817, current row 819, and goal row 821. The data in the baseline row 817 may be entered from the health assessment used to determine the DPP for the consumer 701. The DPP provider 704 can take measurements and review clinical data during one or more initial assessments with the consumer 704. The DPP provider 704 can enter the results from the first meeting(s) into the shared database, which may update the data in the baseline row 817.

The data in the goal row 821 is entered by the DPP provider 704 and can be based on the average results of past participants in the DPP. Alternatively, the DPP provider 704 and the consumer 701 may set goals, which are entered into the shared database by the DPP provider 704. In this example, the baseline row 817 of the consumer dashboard Boo is populated with exemplary data for Patient 1 of FIG. 1. The current row 819 and the goal row 821 of the consumer dashboard Boo are populated demonstrative data to illustrate typical trends of an exemplary patient.

The current row 819 provides the consumer 701 real-time data of the metrics being tracked during the DPP. For example, the consumer 701 can track data and milestones by accessing the current row 819 in the consumer dashboard Boo provided by the integrator 703 (Step 722). The integrator 703 continually updates the data and populates the fields in the current row 819 of the consumer dashboard Boo for viewing by the consumer 701 (Step 723).

In some embodiments, the integrator 703 can send one or more reports regarding the consumer's progress to the medical healthcare provider 702 (Step 724). The report can confirm successful completion of the DPP by the consumer 701 or, alternatively, can report the status if the DPP was not successfully completed. In this way the healthcare provider 702 can report aggregate quality metrics to Medicare/Medicaid agencies and the CDC, as well as chart and report the consumer's performance to the DPP.

In some embodiments, the integrator 703 reserves a portion of the payment from the payer 705, as compensation to the integrator for facilitating and managing the process. Alternatively, the payer 705 may pay a premium over the standard rate for the DPP in order to compensate the integrator 703 facilitating and managing the process. Typically, the premium paid by the payer 705 is less than the cost that the payer 705 would otherwise incur to facilitate and manage the process if the integrator 703 were not used, thus resulting in a net cost saving for the payer 705 in any event. In some embodiments, a set-up fee or a records fee may be included in the claim sent to the payer 705. This fee reimburses the integrator 703 for the costs of enrolling the consumer 701 in the DPP provided by the best-fit DPP provider and initiating an account for the consumer 701.

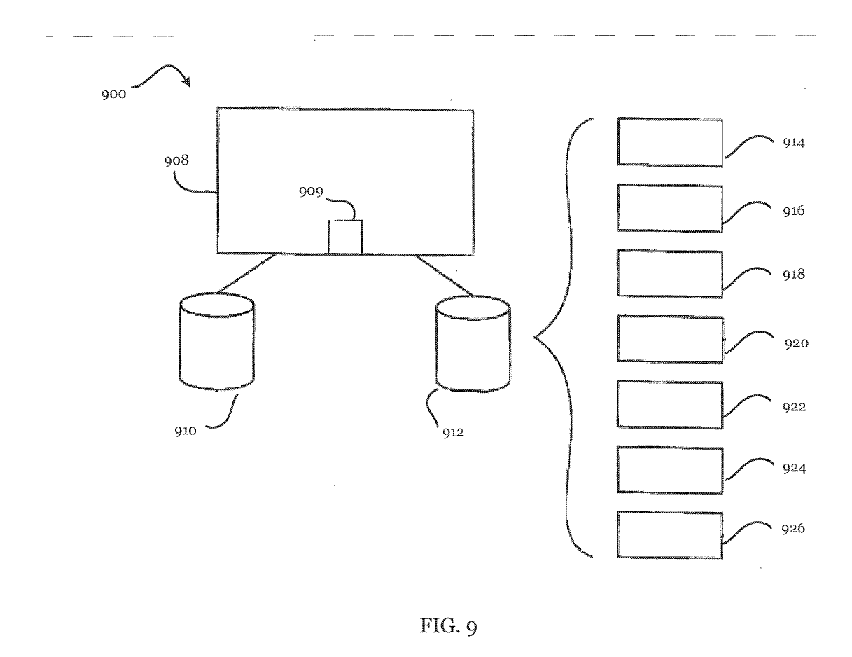

Referring now to FIG. 9, an integrator computer module 908 may be configured to perform any number of the various functions and tasks described herein. For example, a database system 900 includes an integrator computer module 908 having a processor or processing system 909. The integrator computer module 908 can be configured to maintain a first database 510 of DPP providers, and a second database 912 of candidate consumers; that is, the integrator builds and manages a relational database of health plan members. The integrator computer module 908 may be configured to recruit candidate consumers into the database 912 using at least the following sources (also referred to as entry vectors): employers 914, medical providers 916, health system 918, health plans 920, self-referral 922, network providers 924, and CBOs 926.

The foregoing sources may submit aggregate data to the integrator, whereupon the integrator analyses the data to determine eligibility and make program recommendations respecting qualifying consumers.

The integrator computer module 908 may be configured to import data sets from the foregoing vectors, stratify the data, and identify candidate consumers that fit a particular profile or otherwise have a need for one or more DPPs. In contrast, CBOs 926 are not typically equipped with the data security systems and protocols (e.g., HPPA compliant systems) or other processing infrastructure needed to securely manage large data sets.

The integrator computer module 908 can be configured to perform automated triage and patient risk stratification for a population of new candidates, whereupon high-risk candidates may be enrolled into appropriate disease prevention programs.

For example, employers 914 may be brought into the system based on the fact that an employer's benefits package (e.g., an employee health insurance policy) has been revised to cover prevention programs. However, when an employer 914 offers coverage for DPPs and encourages employees to participate in such program, the influx of a population of new candidate consumer can overwhelm resources for enrollment into one or more DPPs. The integrator computer module 908 imports large data sets for the population and performs electronic triage on the data set results during the stratification stage, which assigns each new candidate into a risk category (for example, into a high health risk level, a medium health risk level, or a low health risk level). The integrator computer module 908 may then only use a portion of the data set, which only includes the new candidates in the high health risk level and match these new candidates with best-fit DPP and DPP provider.

If resources are still available for DPPs or after the high risk candidates have gone through the preventative programs, the integrator computer module 908 can use a portion of the data set, which may only include the new candidates in the medium health risk level and match these new candidates with best-fit DPP and DPP provider.

The integrator computer module 908 can be configured to perform triage on a large group of new candidates, which can include the steps of performing a health risk assessment for each new candidate in the group, which results in an assignment of a wellness score for each new candidate in the group; and classifying the group of new candidates into a hierarchy of risk levels based on the wellness score for each candidate. The hierarchy of risk levels can include a high health risk level, a medium health risk, and a low health risk. The triage can prioritize the new candidates in the high health risk level for enrollment in a best-fit DPP and DPP provider.

In an alternate embodiment, candidate consumers may be recruited into the system (i.e., into the integrator's database of candidate consumers) by a health plan, which initiates a call, email, or other communication to a candidate consumers. Those skilled in the art will appreciate that a health plan may be triggered to reach out to candidate consumers by patient costs exceeding a predetermined threshold, an emergency room visit, a claim for payment, an indication that the candidate consumer is not taking medications as prescribed, or another out of profile event or circumstance.

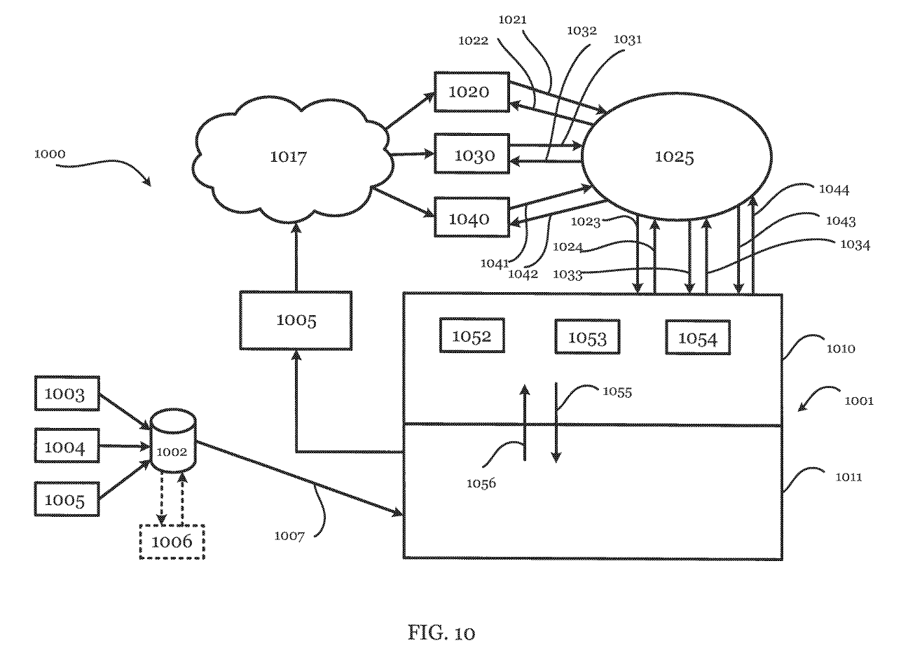

FIG. 10 illustrates a schematic block diagram of an exemplary integrator system 1000, which includes an integrator application engine 1010 configured to run on an integrator computer module 1001.

An integrator computer module 1001 comprises an integrator application engine 1010 and a customer relationship management (“CRM”) system 1011, which may be implemented as a software module. The integrator application engine 1010 can receive 1056 data from the CRM 1011. Data from the integrator application engine 1010 can send 1055 data to populate fields in the CRM 1011.

A database 1002 can be configured to receive data from a plurality of sources including a first source 1003, a second source 1004, and an nth source 1005. The first source 1003, the second source 1004, and the nth source 1005 populate the database 1002 with data from candidate (participants or patients), which can include name, contact information, email address, healthcare provider plan, and demographic data, such as, age, and ethnicity. In some embodiments, the first source 1003, the second source 1004, and the nth source 1005 can be any of the sources described in FIG. 9. The database 1002 operates within the framework of HIPAA and is protected with the appropriate firewalls and other safeguards to protect and limit access to the candidate data.

The data in the database 1002 can be sent thru a scrubbing program 1006, which can be configured to clean the data, fill in missing fields, and/or eliminate duplicates. The scrubbed data 1007 is sent to the CRM 1011 software module operating within the integrator computer module 1001. CRM software modules are well known to those skilled in the art. For example, a Salesforce platform (Salesforce.com, San Francisco, Calif.) can be used as the CRM 1011.

After the scrubbed data 1007 is cataloged by the CRM loll, the CRM 1011 sends a message including an attached weblink or other indicia of an integrator portal, along with at least a portion of the data relating to candidates, to an email distribution module 1015. Email distribution modules are well known to those skilled in the art. For example, a Marketo marketing automation platform (Marketo, Inc., San Mateo, Calif.) can be used as the email distribution module. The email distribution module 1015 sends emails to a plurality of candidates via the cloud 1017 (e.g., the internet). Upon receipt of the email, a candidate is directed to a website 1025 by clicking the weblink embedded in the email.

In this example, Patient 1 1020 is exemplary patient 1 from FIGS. 1-4. Moving back to FIG. 10, Patient 1 1020 opens the email and clicks the weblink, which puts the Patient 1 1020 in contact with the website 1025. Patient 1 1020 inputs data 1021 based on a series of health risk assessment questions and lifestyle preferences, as provided by the website 1025. The received data 1023 from Patient 1 1020 is entered into the integrator application engine 1010 for analysis and health risk stratification. In some embodiments, the integrator application engine 1010 can use machine learning to direct the health risk assessment questions based on the received data 1023. The integrator application engine 1010 analyzes the received data 1023 (which can include the answers to the health risk assessment questions and the survey of personal preferences, as well as physical characteristics such as, age, height, weight, sex, and ethnicity) and designs a personalized precision prevention plan 1052 for Patient 1 1020. As part of the personalized precision prevention plan 1052, a wellness score is generated for the Patient 1 1020.

In this example, the wellness score for Patient 1 1020 is 143 (see FIG. 1), which is a high risk level. The personalized precision prevention plan 1052 would include DPPs for, at least the highest priority chronic diseases 107, which are CHF and CAD (see FIG. 1). However, Patient 1 1020 has high chronic disease risk score (see FIG. 2) for diabetes 223, depression 229, COPD 235, hypertension 241, and hyperlipidemia 247, which are also addressed by the personalized precision prevention plan 1052 (simultaneously (all at once), sequentially, overlapping, or some together and others later in time). In addition, the personalized precision prevention plan 1052 would include DPP programming to change behavior for improvement in the highest priority lifestyle areas 109, which are tobacco, weight management, and physical activity (see FIG. 1).

The personalized precision prevention plan 1052 is communicated 1024 to Patient 1 1020. The integrator application engine 1010 can enroll Patient 1 1020 into one or more DPPs identified by the personalized precision prevention plan 1052 and appropriate notices of enrollment 1022 are sent to the Patient 1 1020 (and optionally to the DPP providers). In one variation of this example, Patient 1 1020 can track progress and/or results for the personalized precision prevention plan 1052 in a dashboard (such as exemplary dashboard Boo illustrated in FIG. 8).

Similarly, Patient 4 1030 is exemplary patient 4 from FIGS. 1-4. Patient 4 1030 interfaces with the website 1025. Patient 4 1030 inputs data 1031 based on the health risk assessment questions and a survey of personal preferences. The received data 1033 from the Patient 4 1030 is entered into the integrator application engine 1010 for analysis and health risk stratification. The integrator application engine 1010 analyzes the received data 1033 and designs a second personalized precision disease prevention plan 1053 for Patient 4 1030. As part of the second personalized precision prevention plan 1053, a wellness score is generated for Patient 4 1030.

In this example, the wellness score for Patient 4 1030 is 66 (see FIG. 1), which is a medium risk level. An exemplary personalized precision prevention plan 1053 may include DPPs for at least the highest priority chronic diseases 107 (e.g., diabetes and depression) (see FIG. 1). However, Patient 4 1030 has high chronic disease risk score (see FIG. 2) for COPD 235, which is also addressed by the personalized precision prevention plan 1053. In addition, the personalized precision prevention plan 1053 would include DPP programming to change behavior for improvement in the highest priority lifestyle areas 109, which are nutrition, weight management, and stress (see FIG. 1).

The second personalized precision prevention plan 1053 is communicated 1034 to Patient 4 1030. The integrator application engine 1010 can enroll Patient 4 1030 into the DPP program or programs associated with the second personalized precision prevention plan 1053 and send appropriate notices of enrollment. Since the answers to the health risk assessment questions, the survey of personal preferences, and physical characteristics will be different for Patient 1 1020 and Patient 4 1030, the personalized precision prevention plan 1052 and the second personalized precision prevention plan 1053 will likely be different and specific to each individual patient.

Continuing with the above example, Patient 8 1040 is exemplary Patient 8 from FIGS. 1-4. Patient 8 1040 interfaces with the website 1025. Patient 8 1040 inputs data 1041 based on the health risk assessment personal preference survey. The received data 643 from the Patient 8 1040 is entered into the integrator application engine 1010 for analysis and health risk stratification. The integrator application engine 610 analyzes the received data 1043 and designs a third personalized precision disease prevention plan 1054 for Patient 8 1040. As part of the third personalized precision prevention plan 1054, a wellness score is generated for the Patient 8 1040.

In this example, the wellness score for Patient 8 1040 is 23 (see FIG. 1), which is a low risk level. The personalized precision prevention plan 1054 would include a DPP the highest priority chronic diseases 107, which is diabetes (see FIG. 1). In addition, the personalized precision prevention plan 1054 would include DPP programming to change behavior for improvement in the highest priority lifestyle areas 109, which is stress (see FIG. 1).

The third personalized precision prevention plan 1054 is communicated 1044 to the Patient 8 1040. The integrator application engine 1010 can enroll the Patient 8 1040 into one or more DPPs identified in the third personalized precision prevention plan 1054, and send a notice of enrollment 1042 along with instructions to the Patient. Since the risk level of the wellness score is low, enrollment in the DPP may be delayed. If enrollment in the DPP is delayed, the system will continue to monitor the wellness score of Patient 8 1040 for changes in risk level and updates the third personalized precision prevention plan 1054, as needed.

Of course the third personalized precision prevention plan 1054, the personalized precision prevention plan 1052, and the second personalized precision prevention plan 1053 will inevitably be different and specific to each individual patient. In addition, the wellness score will inevitably be different and specific to each individual patient.

The integrator system is theoretically infinitely scalable for any the number of patients (consumers). In some embodiments, the integrator system uses series of algorithms and patient health risk stratification to determine one or more “best-fit” programs for a patient, thereby allowing the patient to explore options based on his or her expressed preferences. Successful application of these insights can positively drive program engagement, influence health and wellness behaviors, and support ongoing retention, which significantly increases the probability of successful program completion by the patient.

In this context, “best-fit” implies meaningful engagement to ensure satisfaction of milestones, as well as successful DPP completion. Various examples of DPP programs include, inter alia, the following categories: i) lifestyle/prevention (pre-chronic); ii) chronic disease (e.g., congestive heart failure (“CHF”), coronary artery disease (“CAD”), type-2 diabetes, depression, chronic obstructive pulmonary disease (“COPD”), hypertension, and hyperlipidemia.); iii) behavioral health (e.g., addiction, domestic violence, anger management, depression, anxiety); and iv) pharmaceuticals, including compliance and dosage protocols.

Accommodating individual consumer needs and preferences in this way enables a more personalized approach to care, allowing health plans to engage with their members through prevention, thereby mitigating the higher long term costs of chronic disease treatment.

Various embodiments relate to systems, methods, and on-line platforms for: i) the seamless integration of data among clinical providers (doctors and hospital groups), DPP providers, and Health Plans; ii) providing a sustainable financial model for the DPP providers; iii) a medical record that “lives” in the community and which is dynamically updated by the DPP providers through class instructors, counselors, or coaches); iv) a portal for facilitating the DPP, which can be accessed by candidate patients; and v) various algorithms and patient health risk stratification for determining whether a candidate patient is eligible, qualified, enrolled, and compliant with various DPPs.

In some embodiments, a method of patient risk stratification can include processing a group of clinical factors, lifestyle factors, and medication compliance factors, for each patient of a population; classifying the population of patients into a hierarchy of risk levels; and assigning a health risk status (a wellness score) to each patient of the population, which is based on the group of factors.

The method can include accessing the health risk status of a patient and directing the patient to a DPP, which is designed to improve the patient's health, to establish changes in behavior, and to prevent development of a chronic disease by the patient.

The method can include accessing the health risk status of a patient; accessing medical records of the patient; combining the health risk status and the medical record with demographic and socioeconomic characteristics for the patient; and creating a comprehensive patient profile.

The method can include combining the health risk status and the medical record; and determining a list of chronic diseases that the patient may develop in the next year.

The method can include assigning the patient to a DPP corresponding to one of the chronic diseases on the list.

The method can include mapping the comprehensive patient profile over a group of DPP providers qualified to deliver the DPP; and determining the optimal DPP provider for the patient.

The method can include enrolling the patient in the DPP with the optimal DPP provider.

Some embodiments provide a method for performing automated triage and patient risk stratification for a population of patient, and for enrolling a high-risk patient into a disease prevention program.

The method can include receiving contact from a population of patients; performing a health risk assessment for each patient in the population; sorting data from the health risk assessment into clinical factors, lifestyle factors, and medication compliance factors; calculating a clinical score from the clinical factors for each patient in the population; calculating a lifestyle score from lifestyle factors for each patient in the population; calculating a medication non-compliance score factors from the medication compliance factors for each patient of a population; assigning a wellness score for each patient of a population, wherein the wellness score is a combination of the clinical score, the lifestyle score, and the medication non-compliance score; classifying the population of patients into hierarchy of risk levels based on the wellness score for each patient; and enrolling a high risk patient in a disease prevention program.

In addition, the method can include determining a list of high priority chronic diseases from the clinical factors for the high risk patient; and assigning the high risk patient to the disease prevention program corresponding to one of the chronic diseases on the list.

The method can include accessing the wellness score of the patient; accessing medical records of the patient; combining the wellness score and the medical record with demographic and socioeconomic characteristics for the patient; and creating a comprehensive patient profile.

The method can include mapping the comprehensive patient profile over a group of disease prevention program providers qualified to deliver the disease prevention program; determining the optimal disease prevention program provider for the patient; and enrolling the patient with the optimal disease prevention program provider.

In some embodiments, the disease prevention program is designed to improve the patient's health, to establish changes in behavior, to prevent development of a chronic disease by the patient, and to lower a risk level for the patient.

The method can include surveying the patient for personal preferences for the disease prevent program; creating a matrix of personal preferences for the patient; comparing the matrix of personal preferences for the patient against an ideal matrix of personal preferences for a plurality of providers; and determining a best fit provider for the patient based on the comparing the matrix.

The method can include enrolling the patient with the best fit provider; and providing the patient a notice of enrollment in the disease prevention program with the best-fit provider.

The method can include identifying a location of a residence of the patient; comparing the location patient against a location of the plurality of providers; determining a best fit provider for patient based on the comparing the matrix and the comparing the location; and enrolling the patient with the best fit provider.

The method can include providing the patient a notice of enrollment in the disease prevention program with the best-fit provider.

The method can include invoicing the patient's health insurance plan for a claim to the disease prevention plan; receiving a payment for the claim from the patient's health insurance plan; tracking progress of the patient in the disease prevention program; recognizing a milestone completed by patient in the disease prevention program; and sending a payment to a disease prevention program provider for the milestone in the disease prevention program.

The method can include retaining a portion of the payment for the claim from the patient's health insurance plan.

The payment for the claim from the patient's health insurance plan includes a premium above the payment to the disease prevention program provider for the milestone in the disease prevention program.

The payment for the claim from the patient's health insurance plan includes a records fee for an account for the patient.

The method can include a hierarchy of risk levels, which are high risk level, medium risk level, and low risk level.

The method can include enrolling a medium risk patient in a disease prevention program.

The method can include calculating the number of patients classified in the high risk level; determining a list of high priority chronic diseases from the clinical factors for each of the number of patients classified in the high risk level; ranking the list of high priority chronic diseases by total number of patients for each chronic disease on the list; reviewing an inventory of disease prevention program providers for each chronic disease on the list; and determining if a supply of disease prevention programs is greater than the total number of patients for each chronic disease.

The method can include sorting the population of patients into hierarchy of risk levels based on the wellness score for each patient, wherein the hierarchy of risk levels are high risk level, medium risk level, and low risk level; determining the number of patient in each of the risk levels; determining an average wellness score for the population of patients; and determining an average wellness score for the population of patients for each of the risk levels.

Some embodiments provide a method performed by a computer system for automated processing of patient risk stratification for a population, calculating wellness score for an individual patent, assigning a risk level to the patient and enrolling the patient in a disease prevention program.

Some embodiments provide a computer system for performing automated triage and patient risk stratification for a population of patient, and for enrolling a high-risk patient into a disease prevention program.

The computer system can be configured to perform the steps of: receiving contact from a population of patients; performing a health risk assessment for each patient in the population; sorting data from the health risk assessment into clinical factors, lifestyle factors, and medication compliance factors; calculating a clinical score from the clinical factors for each patient in the population; calculating a lifestyle score from lifestyle factors for each patient in the population; calculating a medication non-compliance score factors from the medication compliance factors for each patient of a population; assigning a wellness score for each patient of a population, wherein the wellness score is a combination of the clinical score, the lifestyle score, and the medication non-compliance score; classifying the population of patients into hierarchy of risk levels based on the wellness score for each patient; and enrolling a high risk patient in a disease prevention program.

The computer system can be further configured to perform determining a list of high priority chronic diseases from the clinical factors for the high risk patient; and assigning the high risk patient to the disease prevention program corresponding to one of the chronic diseases on the list.

Some embodiments provide computer code stored in a non-transient medium for performing, when executed by a computer processor, the steps of: receiving contact from a population of patients; performing a health risk assessment for each patient in the population; sorting data from the health risk assessment into clinical factors, lifestyle factors, and medication compliance factors; calculating a clinical score from the clinical factors for each patient in the population; calculating a lifestyle score from lifestyle factors for each patient in the population; calculating a medication non-compliance score factors from the medication compliance factors for each patient of a population; assigning a wellness score for each patient of a population, wherein the wellness score is a combination of the clinical score, the lifestyle score, and the medication non-compliance score; classifying the population of patients into hierarchy of risk levels based on the wellness score for each patient; and enrolling a high risk patient in a disease prevention program.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

As used herein, the phrase “at least one of A, B, and C” can be construed to mean a logical (A or B or C), using a non-exclusive logical “or,” however, can be contrasted to mean (A, B, and C), in addition, can be construed to mean (A and B) or (A and C) or (B and C). As used herein, the phrase “A, B and/or C” should be construed to mean (A, B, and C) or alternatively (A or B or C), using a non-exclusive logical “or.”

It should be understood that steps within a method may be executed in different order without altering the principles of the present disclosure. For example, various embodiments may be described herein in terms of various functional components and processing steps. It should be appreciated that such components and steps may be realized by any number of hardware components configured to perform the specified functions.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

What is claimed:

1. A computer implemented method for performing automated triage and patient risk stratification for each of a population of patients, and for enrolling a high-risk patient into a disease prevention program, the method comprising:
performing a health risk assessment for each patient;
sorting data from the health risk assessment into clinical factors, lifestyle factors, and medication compliance factors;
calculating a clinical score from the clinical factors for each patient;
calculating a lifestyle score from lifestyle factors for each patient;
calculating a medication non-compliance score from the medication compliance factors for each patient;
assigning a wellness score for each patient, the wellness score comprising a combination of the clinical score, the lifestyle score, and the medication non-compliance score;
classifying each patient into a hierarchy of risk levels including a high risk level based on the wellness scores; and
enrolling a high risk patient in a disease prevention program.

2. The method according to claim 1, further comprising the steps of:
determining a list of high priority chronic diseases from the clinical factors for the high risk patient; and
assigning the high risk patient to the disease prevention program corresponding to one of the chronic diseases on the list.

3. The method according to claim 1, further comprising the steps of:
accessing the wellness score of the patient;
accessing medical records of the patient;
combining the wellness score and the medical record with demographic and socioeconomic characteristics for the patient; and
creating a comprehensive patient profile.

4. The method according to claim 3, further comprising the steps of:
mapping the comprehensive patient profile over a group of disease prevention program providers qualified to deliver the disease prevention program;
determining the optimal disease prevention program provider for the patient; and
enrolling the patient with the optimal disease prevention program provider.

5. The method according to claim 1, wherein the disease prevention program is designed to improve the patient's health, to establish changes in behavior, to prevent development of a chronic disease by the patient, and to lower a risk level for the patient.

6. The method according to claim 1, further comprising the steps of:
surveying the patient for personal preferences for the disease prevent program;
creating a matrix of personal preferences for the patient;
comparing the matrix of personal preferences for the patient against an ideal matrix of personal preferences for a plurality of providers; and
determining a best fit provider for the patient based on the comparing the matrix.

7. The method according to claim 6, further comprising the step of:
enrolling the patient with the best fit provider; and
providing the patient a notice of enrollment in the disease prevention program with the best-fit provider.

8. The method according to claim 6, further comprising the steps of
identifying a location of a residence of the patient;
comparing the location patient against a location of the plurality of providers; and
determining a best fit provider for patient based on the comparing the matrix and the comparing the location; and
enrolling the patient with the best fit provider.

9. The method according to claim 8, further comprising the step of:
providing the patient a notice of enrollment in the disease prevention program with the best-fit provider.

10. The method according to claim 1, further comprising:
invoicing the patient's health insurance plan for a claim to the disease prevention plan;
receiving a payment for the claim from the patient's health insurance plan;
tracking progress of the patient in the disease prevention program;
recognizing a milestone completed by patient in the disease prevention program; and
sending a payment to a disease prevention program provider for the milestone in the disease prevention program.

11. The method according to claim 10, further comprising the step of retaining a portion of the payment for the claim from the patient's health insurance plan.

12. The method according to claim 11, wherein the payment for the claim from the patient's health insurance plan includes a premium above the payment to the disease prevention program provider for the milestone in the disease prevention program.

13. The method according to claim 11, wherein the payment for the claim from the patient's health insurance plan includes a records fee for an account for the patient.

14. The method according to claim 1, wherein the hierarchy of risk levels are high risk level, medium risk level, and low risk level.

15. The method according to claim 14, further comprising the step of enrolling a medium risk patient in a disease prevention program.

16. The method according to claim 14, further comprising the step of:
calculating the number of patients classified in the high risk level;
determining a list of high priority chronic diseases from the clinical factors for each of the number of patients classified in the high risk level;
ranking the list of high priority chronic diseases by total number of patients for each chronic disease on the list;
reviewing an inventory of disease prevention program providers for each chronic disease on the list; and
determining if a supply of disease prevention programs is greater than the total number of patients for each chronic disease.

17. The method according to claim 1, further comprising the steps of:
sorting the population of patients into hierarchy of risk levels based on the wellness score for each patient, wherein the hierarchy of risk levels are high risk level, medium risk level, and low risk level;
determining the number of patient in each of the risk levels;
determining an average wellness score for the population of patients; and
determining an average wellness score for the population of patients for each of the risk levels.

18. A computer system for performing automated triage and patient risk stratification for a population of patient, and for enrolling a high-risk patient into a disease prevention program, the computer system configured to perform the steps of:
receiving contact from a population of patients;
performing a health risk assessment for each patient in the population;
sorting data from the health risk assessment into clinical factors, lifestyle factors, and medication compliance factors;
calculating a clinical score from the clinical factors for each patient in the population;
calculating a lifestyle score from lifestyle factors for each patient in the population;
calculating a medication non-compliance score factors from the medication compliance factors for each patient of a population;
assigning a wellness score for each patient of a population, wherein the wellness score is a combination of the clinical score, the lifestyle score, and the medication non-compliance score;
classifying the population of patients into hierarchy of risk levels based on the wellness score for each patient; and
enrolling a high risk patient in a disease prevention program.

19. The computer system according to claim 18, further configured to perform the steps of:
determining a list of high priority chronic diseases from the clinical factors for the high risk patient; and
assigning the high risk patient to the disease prevention program corresponding to one of the chronic diseases on the list.

20. Computer code stored in a non-transient medium for performing, when executed by a computer processor, the steps of:
receiving contact from a population of patients;
performing a health risk assessment for each patient in the population;
sorting data from the health risk assessment into clinical factors, lifestyle factors, and medication compliance factors;
calculating a clinical score from the clinical factors for each patient in the population;
calculating a lifestyle score from lifestyle factors for each patient in the population;
calculating a medication non-compliance score factors from the medication compliance factors for each patient of a population;
assigning a wellness score for each patient of a population, wherein the wellness score is a combination of the clinical score, the lifestyle score, and the medication non-compliance score;
classifying the population of patients into hierarchy of risk levels based on the wellness score for each patient; and
enrolling a high risk patient in a disease prevention program.
